(19) United States
(12) Patent Application Publication
(10) Pub. No.: US 2026/0295276 A1
(43) Pub. Date: Oct. 1, 2026

(54) TRANSIENT CARDIAC RESYNCHRONIZATION THERAPY

(71) Applicant: Medtronic, Inc., Minneapolis, MN (US)

(72) Inventor: Richard N. Cornelussen, Maastricht (NL)

(21) Appl. No.: 19/479,566

(22) PCT Filed: Apr. 25, 2024
(86) PCT No.: PCT/US2024/026348

§ 371 Date: Oct. 28, 2025

Related U.S. Application Data
(60) Provisional application No. 63/462,689, filed on Apr. 28, 2023.

(51) Int. Cl. A61N 1/368; A61B 5/00; A61B 5/366

(52) U.S. Cl. CPC A61N 1/3688; A61B 5/366; A61B 5/4836; A61B 5/686; A61N 1/3682; A61N 1/36843

(57) ABSTRACT

Illustrative devices and methods may provide cardiac resynchronization therapy (CRT) that utilizes transient CRT settings to mitigate right ventricular load during implementation of the CRT. The transient CRT settings may increase in aggressiveness until reaching target CRT settings. Further, the transient CRT settings may decrease in aggressiveness in response to ventricular deterioration.

### RELATED APPLICATIONS

This application claims priority to U.S. Provisional Patent Application No. 63/462,689, filed on Apr. 28, 2023, which is incorporated by reference herein in its entirety.

This disclosure generally relates to delivery of transient cardiac resynchronization therapy, for example, prior to delivery of cardiac resynchronization therapy.

Performance characteristics of a patient's heart may be evaluated to provide individualized treatment to patients using cardiac resynchronization therapy (CRT). The beat of the heart is controlled by the sinoatrial node, a group of conductive cells located in the right atrium near the entrance of the superior vena cava. A depolarization signal generated by the sinoatrial node activates the atrioventricular node. The atrioventricular node briefly delays the propagation of the depolarization signal, allowing blood in the atria to drain into the ventricles, before passing the depolarization signal to the ventricles of the heart. The coordinated contraction of both ventricles drives the flow of blood through the torso of a patient. In certain circumstances, the conduction of the depolarization signal from the atrioventricular node to the left and right ventricles may be interrupted or slowed. This may result in a dyssynchrony in the contraction of the left and right ventricles, and eventually in heart failure or death.

CRT may correct the symptoms of electrical dyssynchrony by providing pacing therapy to one or both ventricles or atria, e.g., by providing pacing to encourage earlier activation of the left or right ventricles. The ventricles may be paced so as to control the ventricles such that the ventricles contract in synchrony. Many patients undergoing CRT have experienced improved ejection fraction, increased exercise capacity, and an improved feeling of well-being.

Providing CRT to a patient may involve determining whether the patient will derive benefit from the CRT prior to implantation of a cardiac rhythm device. Additionally, detailed evaluations may aid in determining optimal placement of one or more ventricular pacing leads or optimal programming of device parameters, such as selection of electrodes on multi-polar right or left ventricular leads, as well as selection of the timing of the pacing pulses delivered to the electrodes, such as atrioventricular (AV) pacing delay and interventricular (VV) pacing delay.

The working mechanism attributed to CRT may be described as its instantaneous re-coordinating effect on left ventricular electro-mechanical function. Through biventricular pacing, CRT can, at least partially, repair the left ventricular mechanical imbalance by inter-and intra-ventricular resynchronization of the cardiac electric activation. The right ventricle (RV) may not be considered when implementing CRT for a patient. The loading condition of either ventricle, however, directly influences the function (e.g., pumping function) of the other ventricle. Also, systolic dysfunction of the RV, as well as diastolic dysfunction, may be an independent predictor of nonresponse to CRT. Moreover, CRT may instantly increase the work load of the right ventricular myocardium in a patient (e.g., patient with heart failure and left bundle branch block). If the patient's right ventricular function is already comprised prior to therapy, CRT can overburden the RV and its function might deteriorate even further thereby impeding any positive effects of CRT on the left ventricle (LV).

### SUMMARY

The present disclosure may be described as being able to overcome a “sudden” increase in RV workload and aggravating LV function when implementing, or beginning, CRT of a patient by utilizing, or providing, transient, or adaptive, CRT settings processes that transiently modifies the CRT settings to the most optimal settings as determined by conventional CRT such as, for example, adaptive CRT. For example, in one embodiment, the CRT settings may include adaptive atrioventricular and interventricular pacing intervals that start at longer than normally applied AV pacing delays and gradually decreases/adapts the timings to the most optimal timings. Further, for example, in one embodiment, the CRT settings may include multipoint ventricular pacing, which may be gradually enabled. The use of transient CRT settings may allow the right ventricle to adapt and contribute to overall performance including LV performance over time (e.g., a transition time period, a CRT settings ramp period, etc.). The target, or final, CRT settings may be the same or similar to the those currently applied for CRT, which have shown that to be associated with decreased morbidity and mortality. The target CRT settings may be determined automatically using an IMD and/or other operably coupled systems, determined by a physician, and/or provided via network connected system.

It may be described that the present disclosure generally relates to mitigating right ventricular load during implementation of CRT by delivering CRT to the patient's heart according to transient CRT settings that are less aggressive than target CRT settings to partially optimize left ventricular functionality. In other words, CRT therapy may be delivered to a patient according to transient CRT settings, where the transient CRT settings are less aggressive than target CRT settings to only partially optimize left ventricular functionality to mitigate right ventricular load, and the transient CRT settings may be transitioned to the target CRT settings over a transition time period.

Additionally, the present disclosure may be described as providing CRT with transient CRT settings, such as transient atrioventricular and interventricular pacing intervals, in patients having right ventricular dysfunction. The transient CRT settings may transition to target CRT settings that optimize left ventricular functionality and restore mechanical synchrony of the patient's heart by electrically activating the heart in a synchronized manner.

In one or more embodiments, the transient, or adaptive, CRT settings processes may be provided (e.g., only provided) to patients with concomitant RV dysfunction as, for example, detected by echocardiogram, other imaging modalities, invasive hemodynamic evaluation, or electrocardiogramalysis. Additionally, in one or more embodiments, the transient, or adaptive, CRT settings processes can be built in, or on top of, existing CRT timing processes such as, for example, adaptive CRT (aCRT) or CRT efficacy enhancements (CRTEE). Further, it may be described that the transient, or adaptive, CRT settings processes can be personalized, accessible to implement, used without additional power consumption or battery usage, and increase the CRT responder rate. Still further, in one or more embodiments, the transient, or adaptive, CRT settings may be used in conjunction with cardiac conduction system pacing therapy modalities such as, for example, left bundle branch pacing therapy, right bundle branch pacing therapy, left ventricular septal pacing therapy, HIS bundle pacing therapy, and any combination of such therapies (e.g., left bundle branch-optimized CRT and HIS-optimized CRT), which may be important as cardiac conduction system pacing therapy may be more effective than traditional myocardial pacing therapies resulting a greater ventricular workload shift than traditional myocardial pacing therapies.

The illustrative devices and methods may be described as including or utilizing temporal adaptive CRT processes to conserve RV function and increase CRT response. To do so, the devices and methods may include determining whether a patient is CRT eligible (for example, whether a patient would benefit from CRT therapy), assessing the patient's baseline RV function, determining whether the patient has RV dysfunction based on the assessment, an providing transient, or temporal, adaptive atrioventricular and/or interventricular pacing intervals. The devices and methods may include assessing the patient's baseline RV function after an assessment time period (such as, for example, a week or more than one week), determining whether the patient's RV dysfunction is better, worse, or absent, and adjusting the providing transient, or temporal, adaptive atrioventricular and/or interventricular pacing intervals based on whether the patient's RV dysfunction is better, worse, or absent.

One illustrative implantable medical device may include an electrode apparatus comprising one or more electrodes configured to deliver cardiac resynchronization therapy (CRT) to a patient's heart using CRT settings and to sense electrical activity of the patient's heart and a computing apparatus operably coupled to the electrode apparatus and comprising processing circuitry. In one embodiment, the computing apparatus may be configured to provide target CRT settings configured to optimize left ventricular functionality and restore mechanical synchrony of the patient's heart by electrically activating the heart in a synchronized manner and mitigate right ventricular load during implementation of CRT by delivering CRT to the patient's heart using the electrode apparatus according to transient CRT settings that are less aggressive than the target CRT settings to partially optimize left ventricular functionality. In one embodiment, the computing apparatus may be configured to deliver CRT according to transient CRT settings to the patient's heart using the electrode apparatus where the transient CRT settings are less aggressive than target CRT settings to partially optimize left ventricular functionality to mitigate right ventricular load and transition from the transient CRT settings to the target CRT settings over a transition time period.

One illustrative method may include delivering cardiac resynchronization therapy (CRT) to a patient's heart using one or more electrodes of an implantable medical device, providing target CRT settings configured to optimize left ventricular functionality and restore mechanical synchrony of the patient's heart by electrically activating the heart in a synchronized manner, and mitigating right ventricular load during implementation of CRT by delivering CRT to the patient's heart using the electrode apparatus according to transient CRT settings that are less aggressive than the target CRT settings to partially optimize left ventricular functionality.

One illustrative method may include delivering cardiac resynchronization therapy (CRT) to a patient's heart using one or more electrodes of an implantable medical device according to transient CRT settings to the patient's heart using the electrode apparatus where the transient CRT settings are less aggressive than target CRT settings to partially optimize left ventricular functionality to mitigate right ventricular load and transitioning from the transient CRT settings to the target CRT settings over a transition time period.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a diagram of an illustrative system including an implantable medical device (IMD) and a programmer.

FIG. 2 is a diagram of the illustrative IMD of FIG. 1.

FIG. 3 is a block diagram of the IMD of FIGS. 1-2.

FIG. 4 is a diagram of an illustrative programmer of the system of FIG. 1.

FIG. 5 is a diagram of an illustrative system including the IMD and programmer of FIG. 1 and additional devices coupled thereto via a network.

FIG. 6 is an illustrative method of mitigating right ventricular load during implementation of CRT, for example, using the system and devices of FIGS. 1-5.

FIG. 7 is a graph of a transient CRT settings over time.

### DETAILED DESCRIPTION

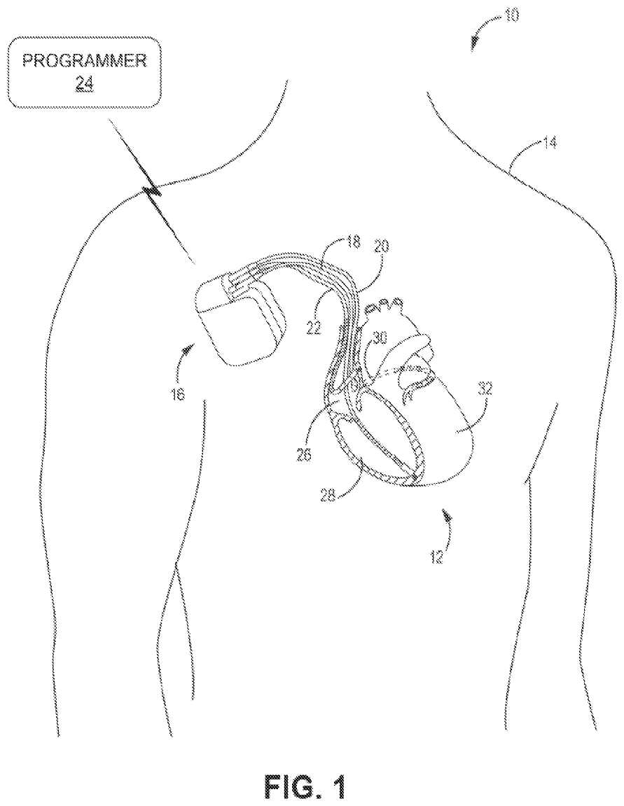

The techniques of this disclosure generally relate to mitigating right ventricular load during implementation of CRT that may be delivered using implantable medical devices (IMDs). Illustrative systems, devices, methods, and processes that are used to mitigate right ventricular load during implementation of CRT are described herein with respect to FIGS. 1-7.

FIG. 1 is a conceptual diagram of an exemplary therapy system 10 that may be configured to deliver pacing therapy, such as cardiac pacing therapy and cardiac resynchronization therapy (CRT), to a patient 14. While the patient 14 is shown as a human, the patient 14 may also be a variety of other types of animals. The therapy system 10 may include an implantable medical device 16 (IMD), which may be coupled to leads 18, 20, 22, and a programmer 24. The IMD 16 may be, e.g., an implantable pacemaker, cardioverter, and/or defibrillator, that delivers, or provides, electrical signals (e.g., paces, etc.) to the heart 12 of the patient 14 via electrodes coupled to one or more of the leads 18, 20, 22, senses electrical signals from the heart 12 of the patient 14 via electrodes coupled to one or more of the leads 18, 20, 22, and/or senses mechanical activity (e.g., sounds, motions, vibrations, etc.) of the heart 12 of the patient 14 via a mechanical heart activity sensor.

The leads 18, 20, 22 extend into the heart 12 of the patient 14 to sense electrical activity of the heart 12 and/or to deliver electrical stimulation to the heart 12. In the example shown in FIG. 1, the right ventricular (RV) lead 18 extends through one or more veins (not shown), the superior vena cava (not shown), and the right atrium 26, and into the right ventricle 28. The left ventricular (LV) coronary sinus lead 20 extends through one or more veins, the vena cava, the right atrium 26, and into the coronary sinus 30 to a region adjacent to the free wall of the left ventricle 32 of the heart 12. The right atrial (RA) lead 22 extends through one or more veins and the vena cava, and into the right atrium 26 of the heart 12.

The IMD 16 may sense, among other things, electrical signals attendant to the depolarization and repolarization of the heart 12 via electrodes coupled to at least one of the leads 18, 20, 22 and mechanical heart activity signals of the heart 12 using a mechanical heart activity sensor. In some examples, the IMD 16 provides pacing therapy (e.g., pacing pulses) to the heart 12 based on the electrical signals sensed within the heart 12. The IMD 16 may be operable to adjust one or more parameters associated with the pacing therapy such as, e.g., pacing rate, R-R interval, AV pacing interval (or delay), VV pacing interval (or delay), and other various timings, pulse width, amplitude, voltage, burst length, etc. Further, the IMD 16 may be operable to use various electrode configurations to deliver pacing therapy, which may be unipolar, bipolar, quadripolar, or further multipolar. Hence, a multipolar lead system may provide, or offer, multiple electrical vectors to pace from. A pacing vector may include at least one cathode, which may be at least one electrode located on at least one lead, and at least one anode, which may be at least one electrode located on at least one lead (e.g., the same lead, or a different lead) and/or on the casing, or can, of the IMD, or electrode apparatus. While improvement in cardiac function as a result of the pacing therapy may primarily depend on the cathode, the electrical parameters like impedance, pacing threshold voltage, current drain, longevity, etc. may be more dependent on the pacing vector, which includes both the cathode and the anode. The IMD 16 may also provide defibrillation therapy and/or cardioversion therapy via electrodes located on at least one of the leads 18, 20, 22. Further, the IMD 16 may detect arrhythmia of the heart 12, such as fibrillation of the ventricles 28, 32, and deliver defibrillation therapy to the heart 12 in the form of electrical pulses. In some examples, IMD 16 may be programmed to deliver a progression of therapies, e.g., pulses with increasing energy levels, until a fibrillation of the heart 12 is stopped.

In some examples, the programmer 24 may be a mobile computing device (such as a smartphone) or a computer workstation. The programmer 24 may include a user interface that receives input from a user. The user interface may include, for example, a keypad and a display, which may, for example, be a liquid crystal display (LCD) or light emitting diode (LED) display. The keypad may take the form of an alphanumeric keypad or a reduced set of keys associated with particular functions. The programmer 24 can additionally or alternatively include a peripheral pointing device, such as a mouse, via which a user may interact with the user interface. In some embodiments, a display of the programmer 24 may include a touch screen display, and a user may interact with the programmer 24 via the display.

A user, such as a physician, technician, patient, or other user, may interact with the programmer 24 to communicate with the IMD 16. For example, a user may interact with the programmer 24 to retrieve physiological or diagnostic information from the IMD 16. A user may also interact with the programmer 24 to program the IMD 16, e.g., select values for operational parameters of the IMD.

Further, for example, a user may use the programmer 24 to retrieve information from the IMD 16 regarding other sensed physiological or diagnostic parameters of the patient 14 such as, for example, right ventricular dysfunction, left ventricular dysfunction, intracardiac or intravascular pressure, activity, posture, respiration, or thoracic impedance. As another example, the user may use the programmer 24 to retrieve information from the IMD 16 regarding the performance or integrity of the IMD 16 or other components of the system 10, such as the leads 18, 20, and 22, or a power source of the IMD 16.

A user may use the programmer 24 to review information from the IMD 16. In some examples, a user may activate features of the IMD 16 by entering a single command via the programmer 24, such as depression of a single key or combination of keys of a keypad or a single point-and-select action with a pointing device.

The IMD 16 and the programmer 24 may communicate via wireless communication using any techniques known in the art. Examples of communication techniques may include, for example, low frequency or radiofrequency (RF) telemetry, but other techniques are also contemplated. In some examples, the programmer 24 may include a programming head that may be placed proximate to the patient's body near the IMD 16 implant site in order to improve the quality or security of communication between the IMD 16 and the programmer 24.

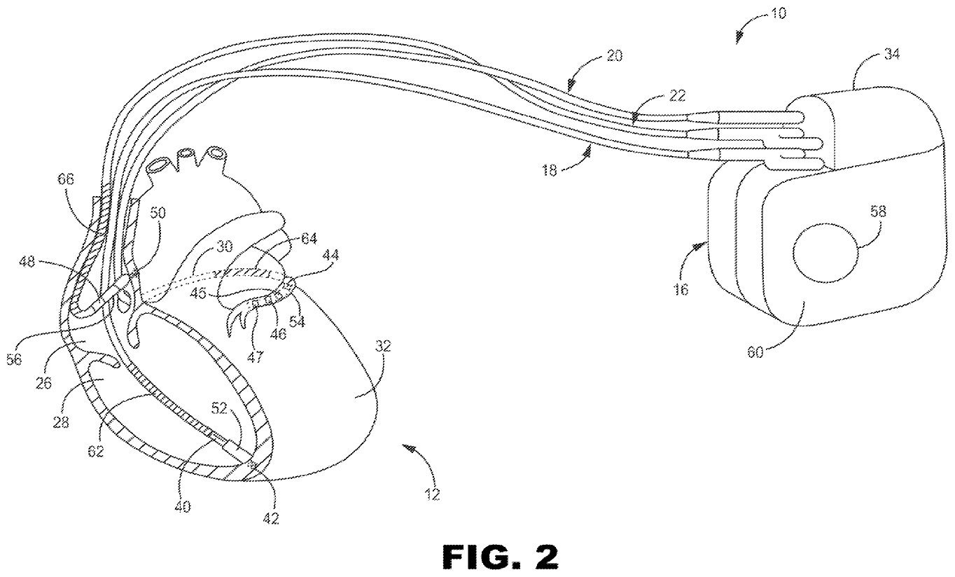

FIG. 2 is a conceptual diagram of the IMD 16 and the leads 18, 20, 22 of therapy system 10 of FIG. 1 in more detail. The leads 18, 20, 22 may be electrically coupled to a therapy delivery module (e.g., for delivery of CRT), a sensing module (e.g., for sensing one or more signals from one or more electrodes), and/or any other modules of the IMD 16 via a connector block 34. In some examples, the proximal ends of the leads 18, 20, 22 may include electrical contacts that electrically couple to respective electrical contacts within the connector block 34 of the IMD 16. In addition, in some examples, the leads 18, 20, 22 may be mechanically coupled to the connector block 34 with the aid of set screws, connection pins, or another suitable mechanical coupling mechanism.

Each of the leads 18, 20, 22 includes an elongated insulative lead body, which may carry a number of conductors (e.g., concentric coiled conductors, straight conductors, etc.) separated from one another by insulation (e.g., tubular insulative sheaths). In the illustrated example, bipolar electrodes 40, 42 are located proximate to a distal end of the lead 18. In addition, bipolar electrodes 44, 45, 46, 47 are located proximate to a distal end of the lead 20 and bipolar electrodes 48, 50 are located proximate to a distal end of the lead 22.

The electrodes 40, 44, 45, 46, 47, 48 may take the form of, or define, ring electrodes, and the electrodes 42, 50 may take the form of, or define, extendable helix tip electrodes mounted retractably within the insulative electrode heads 52, 54, 56, respectively. Each of the electrodes 40, 42, 44, 45, 46, 47, 48, 50 may be electrically coupled to a respective one of the conductors (e.g., coiled and/or straight) within the lead body of its associated lead 18, 20, 22, and thereby coupled to a respective one of the electrical contacts on the proximal end of the leads 18, 20, 22.

The electrodes 40, 42, 44, 45, 46, 47, 48, 50 may further be used to sense electrical signals (e.g., morphological waveforms within electrograms (EGM)) attendant to the depolarization and repolarization of the heart 12. The electrical signals are conducted to the IMD 16 via the respective leads 18, 20, 22. In some examples, the IMD 16 may also deliver pacing pulses via the electrodes 40, 42, 44, 45, 46, 47, 48, 50 to cause depolarization of cardiac tissue of the patient's heart 12. In some examples, as illustrated in FIG. 2, the IMD 16 includes one or more housing electrodes, such as housing electrode 58, which may be formed integrally with an outer surface of a housing 60 (e.g., hermetically sealed housing) of the IMD 16 or otherwise coupled to the housing 60. Any of the electrodes 40, 42, 44, 45, 46, 47, 48, 50 may be used for unipolar sensing or pacing in combination with the housing electrode 58. It is generally understood by those skilled in the art that other electrodes can also be selected to define, or be used for, pacing and sensing vectors. Further, any of electrodes 40, 42, 44, 45, 46, 47, 48, 50, 58, when not being used to deliver pacing therapy, may be used to sense electrical activity during pacing therapy.

As described in further detail with reference to FIG. 2, the housing 60 may enclose a therapy delivery module that may include a stimulation generator for generating cardiac pacing pulses and defibrillation or cardioversion shocks, as well as a sensing module for monitoring the electrical signals of the patient's heart (e.g., the patient's heart rhythm). The leads 18, 20, 22 may also include elongated electrodes 62, 64, 66, respectively, which may take the form of a coil. The IMD 16 may deliver defibrillation shocks to the heart 12 via any combination of the elongated electrodes 62, 64, 66 and the housing electrode 58. The electrodes 58, 62, 64, 66 may also be used to deliver cardioversion pulses to the heart 12. Further, the electrodes 62, 64, 66 may be fabricated from any suitable electrically conductive material, such as, but not limited to, platinum, platinum alloy, and/or other materials known to be usable in implantable defibrillation electrodes. Since electrodes 62, 64, 66 are not generally configured to deliver pacing therapy, any of electrodes 62, 64, 66 may be used to sense electrical activity and may be used in combination with any of electrodes 40, 42, 44, 45, 46, 47, 48, 50, 58. In at least one embodiment, the RV elongated electrode 62 may be used to sense electrical activity of a patient's heart during the delivery of pacing therapy (e.g., in combination with the housing electrode 58, or defibrillation electrode-to-housing electrode vector).

The above-described configuration of the therapy system 10 is merely one example. In other examples, the therapy system may include epicardial leads and/or patch electrodes instead of, or in addition to, the transvenous leads 18, 20, 22 illustrated in FIG. 1. In further embodiments, the therapy system 10 may be implanted in/around the cardiac space without transvenous leads (e.g., leadless/wireless pacing systems) or with leads implanted (e.g., implanted transvenously or using approaches) into the left chambers of the heart (in addition to or replacing the transvenous leads placed into the right chambers of the heart as illustrated in FIG. 1). Further, in one or more embodiments, the IMD 16 may not be implanted within the patient 14. For example, the IMD 16 may deliver various cardiac therapies to the heart 12 via percutaneous leads that extend through the skin of the patient 14 to a variety of positions within or outside of the heart 12. In one or more embodiments, the system 10 may utilize wireless pacing (e.g., using energy transmission to the intracardiac pacing component(s) via ultrasound, inductive coupling, RF, etc.) and sensing cardiac activation using electrodes on the can/housing and/or on subcutaneous leads.

Other example therapy systems that provide electrical stimulation therapy to the heart 12 may include any suitable number of leads coupled to the IMD 16, and each of the leads may extend to any location within or proximate to the heart 12. Such other therapy systems may include three transvenous leads located as illustrated in FIGS. 1-2. Still further, therapy systems may include a single lead that extends from the IMD 16 into the right atrium 26 or two leads that extend into a respective one of the right atrium 26 and the left atrium. In one example, the IMD 16, as a cardiac resynchronization therapy (CRT) device with a left ventricular (LV) lead, may be useful for a HFpEF patient if there is a complete AV node block as a LV lead can be more beneficial than a RV lead in such patients.

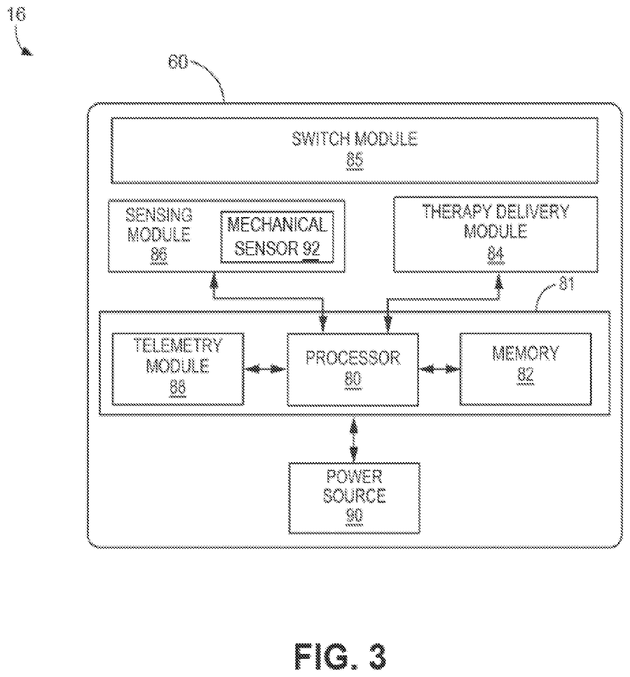

FIG. 3 is a functional block diagram of an illustrative configuration of the IMD 16. As shown, the IMD 16 may include a control module 81, a therapy delivery module 84 (e.g., which may include a stimulation generator), a sensing module 86, and a power source 90. The control module, or apparatus, 81 may include a computing apparatus 80, memory 82, and a telemetry module, or apparatus, 88. The memory 82 may include computer-readable instructions that, when executed, e.g., by the computing apparatus 80, cause the IMD 16 and/or the control module 81 to perform various functions attributed to the IMD 16 and/or the control module 81 described herein. Further, the memory 82 may include any volatile, non-volatile, magnetic, optical, and/or electrical media, such as a random-access memory (RAM), read-only memory (ROM), non-volatile RAM (NVRAM), electrically erasable programmable ROM (EEPROM), flash memory, and/or any other digital media.

The computing apparatus 80 of the control module 81 may include any one or more of a microprocessor, a controller, a digital signal processor (DSP), an application specific integrated circuit (ASIC), a field-programmable gate array (FPGA), and/or equivalent discrete or integrated logic circuitry. In some examples, the computing apparatus 80 may include multiple components, such as any combination of one or more microprocessors, one or more controllers, one or more DSPs, one or more ASICs, and/or one or more FPGAs, as well as other discrete or integrated logic circuitry. The functions attributed to the computing apparatus 80 herein may be embodied as software, firmware, hardware, or any combination thereof.

The control module 81 may be configured to perform one or more methods and processes described herein within respect to assessment, or determination, of RV and/or LV dysfunction, determination, or generation, of target CRT settings, determination, or generation, of transient CRT settings, implantation of transient CRT settings, and changing (e.g., increasing or decreasing) aggressiveness of the transient CRT settings over time or in response to RV and/or LV dysfunction. Further, the control module 81 may control the therapy delivery module, or apparatus, 84 to deliver therapy (e.g., cardiac resynchronization therapy, adaptive pacing, bradycardia pacing, etc.) to the heart 12 according to a selected one or more therapy programs, which may be stored in the memory 82, and based on algorithms, or methods, described further below. More specifically, the control module 81 (e.g., the computing apparatus 80) may control various parameters of the electrical stimulus delivered by the therapy delivery module 84 such as, e.g., AV pacing intervals (or delays), VV pacing intervals (or delays), pacing pulses with the amplitudes, pulse widths, frequency, or electrode polarities, etc., which may be specified by one or more selected therapy programs (e.g., adaptive pacing therapy program, transient CRT settings, target CRT settings, adjustment, and/or modifications programs, pacing therapy programs, pacing recovery programs, capture management programs, etc.). As shown, the therapy delivery module 84 is electrically coupled to electrodes 40, 42, 44, 45, 46, 47, 48, 50, 58, 62, 64, 66, e.g., via conductors of the respective lead 18, 20, 22, or, in the case of housing electrode 58, via an electrical conductor disposed within housing 60 of IMD 16. Therapy delivery module 84 may be configured to generate and deliver electrical stimulation therapy such as pacing therapy to the heart 12 using one or more of the electrodes 40, 42, 44, 45, 46, 47, 48, 50, 58, 62, 64, 66.

For example, the therapy delivery module 84 may deliver pacing stimulus (e.g., pacing pulses) via ring electrodes 40, 44, 45, 46, 47, 48 coupled to leads 18, 20, 22 and/or helical tip electrodes 42, 50 of leads 18, 22. Further, for example, therapy delivery module 84 may deliver defibrillation shocks to the heart 12 via at least two of electrodes 58, 62, 64, 66. In some examples, therapy delivery module 84 may be configured to deliver pacing, cardioversion, or defibrillation stimulation in the form of electrical pulses. In other examples, therapy delivery module 84 may be configured to deliver one or more of these types of stimulation in the form of other signals, such as sine waves, square waves, and/or other substantially continuous time signals.

The IMD 16 may further include a switch module, or apparatus, 85 and the control module 81 (e.g., the computing apparatus 80) may use the switch module 85 to select, e.g., via a data/address bus, which of the available electrodes are used to deliver therapy such as pacing pulses for pacing therapy, or which of the available electrodes are used for sensing. The switch module 85 may include a switch array, switch matrix, multiplexer, or any other type of switching device suitable to selectively couple the sensing module, or apparatus, 86 and/or the therapy delivery module 84 to one or more selected electrodes.

More specifically, the therapy delivery module 84 may include a plurality of pacing output circuits. Each pacing output circuit of the plurality of pacing output circuits may be selectively coupled, e.g., using the switch module 85, to one or more of the electrodes 40, 42, 44, 45, 46, 47, 48, 50, 58, 62, 64, 66 (e.g., a pair of electrodes for delivery of therapy to a bipolar or multipolar pacing vector). In other words, each electrode can be selectively coupled to one of the pacing output circuits of the therapy delivery module using the switch module 85.

The sensing module 86 is coupled (e.g., electrically coupled) to sensing apparatus, which may include, among additional sensing apparatus, the electrodes 40, 42, 44, 45, 46, 47, 48, 50, 58, 62, 64, 66 to monitor electrical activity of the heart 12, e.g., electrocardiogram (ECG)/electrogram (EGM) signals, etc. The ECG/EGM signals may be used to measure or monitor activation times (e.g., ventricular activations times, etc.), heart rate (HR), heart rate variability (HRV), heart rate turbulence (HRT), deceleration/acceleration capacity, deceleration sequence incidence, T-wave alternans (TWA), P-wave to P-wave intervals (also referred to as the P-P intervals or A-A intervals), R-wave to R-wave intervals (also referred to as the R-R intervals or V-V intervals), P-wave to QRS complex intervals (also referred to as the P-R intervals, A-V intervals, or P-Q intervals), QRS-complex morphology, QRS complex intervals, or QRS duration, ST segment (i.e., the segment that connects the QRS complex and the T-wave), T-wave changes, QT intervals, electrical vectors, etc.

The sensing module 86 may further include a mechanical heart activity sensor 92 configured to monitor mechanical activity of the patient's heart 12. The mechanical activity of the patient's heart may include or be representative of one or more motion, movement, sounds, and vibrations of the patient's heart 12 and one or more portions or anatomical mechanisms of the patient's heart 12. For example, the mechanical heart activity sensor 92 may be configured to monitor mechanical activity corresponding to or indicative of closure of the atrioventricular valves (i.e., the mitral valve and the tricuspid valve), closure of the semilunar valves (i.e., the aortic valve and the pulmonary valve), chamber fillings, chamber contractions, and transitions from rapid to slow filling.

It is to be understood that, although the mechanical heart activity sensor 92 is depicted as being part of the sensing module 86 within the housing 60 of the IMD 16, the mechanical heart activity sensor 92 may be external to the housing 60 such as, e.g., part of or included within one of the leads 18, 20, 22, and positioned in various locations within or about the patient's heart 12. Further, in at least one embodiment, the IMD 16 may be a leadless IMD including the mechanical heart activity sensor 92 therein and may be positioned within a chamber of the patient's heart 12 thereby placing the mechanical heart activity sensor 92 within the chamber of the patient's heart 12.

The switch module 85 may also be used with the sensing module 86 to select which of the available electrodes are used, or enabled, to, e.g., sense electrical activity of the patient's heart (e.g., one or more electrical vectors of the patient's heart using any combination of the electrodes 40, 42, 44, 45, 46, 47, 48, 50, 58, 62, 64, 66). Likewise, the switch module 85 may also be used with the sensing module 86 to select which of the available electrodes are not to be used (e.g., disabled) to, e.g., sense electrical activity of the patient's heart (e.g., one or more electrical vectors of the patient's heart using any combination of the electrodes 40, 42, 44, 45, 46, 47, 48, 50, 58, 62, 64, 66), etc. In some examples, the control module 81 may select the electrodes that function as sensing electrodes via the switch module within the sensing module 86, e.g., by providing signals via a data/address bus.

In some examples, sensing module 86 includes a channel that includes an amplifier with a relatively wider pass band than the R-wave or P-wave amplifiers. Signals from the selected sensing electrodes may be provided to a multiplexer, and thereafter converted to multi-bit digital signals by an analog-to-digital converter for storage in memory 82, e.g., as an electrogram (EGM). In some examples, the storage of such EGMs in memory 82 may be under the control of a direct memory access circuit.

In some examples, the control module 81 may operate as an interrupt-driven device and may be responsive to interrupts from pacer timing and control module, where the interrupts may correspond to the occurrences of sensed P-waves and R-waves and the generation of cardiac pacing pulses. Any mathematical calculations may be performed by the computing apparatus 80 and any updating of the values or intervals controlled by the pacer timing and control module may be executed, or take place, following such interrupts. A portion of memory 82 may be configured as a plurality of recirculating buffers, capable of holding one or more series of measured intervals, which may be analyzed by, e.g., the computing apparatus 80 in response to the occurrence of a pace or sense interrupt to determine whether the patient's heart 12 is presently exhibiting atrial or ventricular tachyarrhythmia.

The computing apparatus 80 of IMD 16 may be configured to evaluate, or assess, RV and/or LV dysfunction through monitoring one or more heart metrics indicative of right ventricular and/or left ventricular function. For example, QRS duration may be monitored, which may be used to determine RV dysfunction. Further, the computing apparatus 80 of IMD 16 may detect a tachyarrhythmia episode, such as a ventricular fibrillation, ventricular tachycardia, fast ventricular tachyarrhythmia episode, or a NST episode, based on electrocardiographic activity of heart 12 that is monitored via sensing module 86. For example, sensing module 86, with the aid of at least some of the electrodes 40, 42, 44, 46, 48, 50, 58, 62, 64, and 66 (shown in FIGS. 1-2), may generate an electrocardiogram (ECG) or electrogram (EGM) signal that indicates the electrocardiographic activity. Alternatively, sensing module 86 may be coupled to sense electrodes that are separate from the stimulation electrodes that deliver electrical stimulation to heart 12 (shown in FIGS. 1-2), and may be coupled to one or more different leads than leads 18, 20, 22 (shown in FIGS. 1-2). The ECG signal may be indicative of the depolarization of heart 12.

For example, as previously described, in some examples, processor 80 may identify the presence of a tachyarrhythmia episode by detecting a threshold number of tachyarrhythmia events (e.g., R-R or P-P intervals having a duration less than or equal to a threshold). In some examples, the computing apparatus 80 may also identify the presence of the tachyarrhythmia episode by detecting a variable coupling interval between the R-waves of the heart signal.

The telemetry module 88 of the control module 81 may include any suitable hardware, firmware, software, or any combination thereof for communicating with another device, such as a programmer (such as, for example, a mobile computing devices or smartphones). For example, under the control of the computing apparatus 80, the telemetry module 88 may receive downlink telemetry from and send uplink telemetry to a programmer or mobile computing device with the aid of an antenna, which may be internal and/or external. The computing apparatus 80 may provide the data to be uplinked to a programmer or a mobile computing device and the control signals for the telemetry circuit within the telemetry module 88, e.g., via an address/data bus. In some examples, the telemetry module 88 may provide received data to the computing apparatus 80 via a multiplexer.

The various components of the IMD 16 are further coupled to a power source 90, which may include a rechargeable or non-rechargeable battery. A non-rechargeable battery may be selected to last for several years, while a rechargeable battery may be inductively charged from an external device, e.g., on a daily or weekly basis.

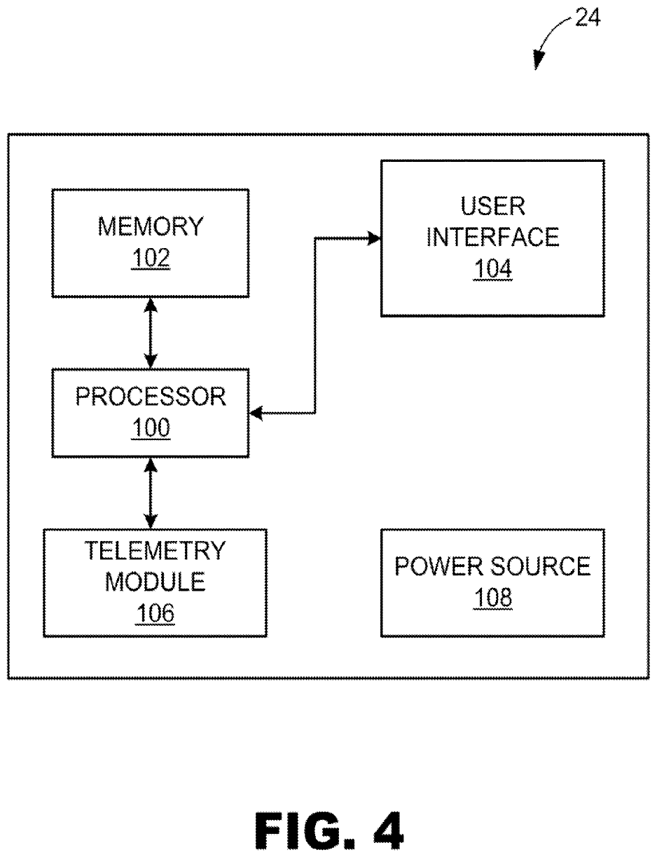

FIG. 4 is a block diagram of an illustrative programmer 24. As shown in FIG. 4, the programmer 24 includes a processor 100, a memory 102, a user interface 104, a telemetry module 106, and a power source 108. The programmer 24 may be a dedicated hardware device with dedicated software for programming of IMD 16. Alternatively, the programmer 24 may be an off-the-shelf computing device (e.g., mobile compute device such as a smartphone) running an application that enables programmer 24 to program IMD 16.

A user may use the programmer 24 to display and review assessment or metrics related to the RV and/or LV dysfunction of the patient's heart, transient CRT settings, and target CRT settings. Additionally, a user may use the programmer 24 to select CRT settings such as, for example, transient CRT settings and target CRT settings, increase or decrease aggressiveness of CRT settings, select therapy programs (e.g., sets of stimulation parameters), generate new therapy programs, modify therapy programs through individual or global adjustments or transmit the new programs to a medical device, such as the IMD 16 of FIG. 1. A user may interact with the programmer 24 via the user interface 104, which may include display to present graphical user interface to a user, and a keypad or another mechanism for receiving input from a user.

The processor 100 can take the form one or more microprocessors, DSPs, ASICs, FPGAs, programmable logic circuitry, or the like, and the functions attributed to processor 100 herein may be embodied as hardware, firmware, software or any combination thereof. The memory 102 may store instructions that cause processor 100 to provide the functionality ascribed to the programmer 24 herein, and information used by processor 100 to provide the functionality ascribed to the programmer 24 herein. The memory 102 may include any fixed or removable magnetic, optical, or electrical media, such as RAM, ROM, CD-ROM, hard or floppy magnetic disks, EEPROM, or the like. The memory 102 may also include a removable memory portion that may be used to provide memory updates or increases in memory capacities. A removable memory may also allow IMD and/or patient data to be easily transferred to another computing device, or to be removed before the programmer 24 is used to program therapy for another patient. The memory 102 may also store information that controls therapy delivery by the IMD 16, such as stimulation parameter values.

The programmer 24 may communicate wirelessly with the IMD 16, such as using RF communication or proximal inductive interaction. This wireless communication is possible through the use of the telemetry module 106, which may be coupled to an internal antenna or an external antenna. An external antenna that is coupled to programmer 24 may correspond to the programming head that may be placed over the heart 12, as described above with reference to FIG. 1. The telemetry module 106 may be similar to telemetry module 88 of the IMD 16 of FIG. 3.

The telemetry module 106 may also be configured to communicate with another computing device via wireless communication techniques, or direct communication through a wired connection. Examples of local wireless communication techniques that may be employed to facilitate communication between the programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with the programmer 24 without needing to establish a secure wireless connection.

The power source 108 delivers operating power to the components of programmer 24 and may include a battery and a power generation circuit to produce the operating power. In some embodiments, the battery may be rechargeable to allow extended operation. Recharging may be accomplished by electrically coupling power source 108 to a cradle or plug that is connected to an alternating current (AC) outlet. In addition or alternatively, recharging may be accomplished through proximal inductive interaction between an external charger and an inductive charging coil within programmer 24. In other embodiments, traditional batteries (e.g., nickel cadmium or lithium-ion batteries) may be used. In addition, programmer 24 may be directly coupled to an alternating current outlet to power the programmer 24. The power source 108 may include circuitry to monitor power remaining within a battery. In this manner, a user interface 104 may provide a current battery level indicator or low battery level indicator when the battery needs to be replaced or recharged. In some cases, power source 108 may be capable of estimating the remaining time of operation using the current battery.

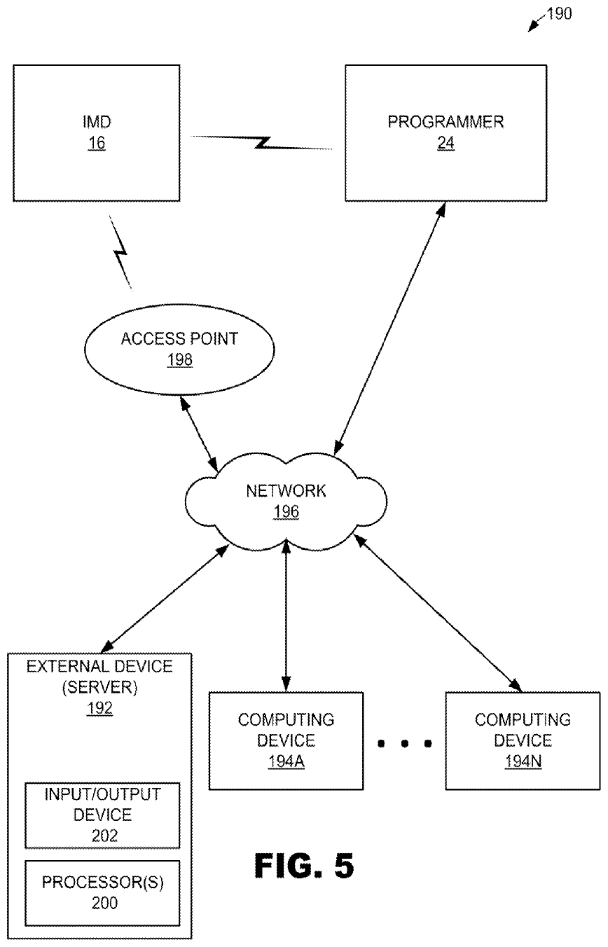

FIG. 5 is a block diagram illustrating a system 190 that includes an external device 192, such as a server, and one or more computing devices 194a-194n that are coupled to the IMD 16 and the programmer 24 shown in FIGS. 1-4 via a network 196, according to one embodiment. In this embodiment, the IMD 16 may use its telemetry module 88 to communicate with the programmer 24 via a first wireless connection, and to communicate with an access point 198 via a second wireless connection. In the example of FIG. 5, the access point 198, the programmer 24, the external device 192, and the computing devices 194a-194n are interconnected, and able to communicate with each other, through a network 196. In some cases, one or more of the access point 198, the programmer 24, the external device 192, and the computing devices 194a-194n may be coupled to the network 196 through one or more wireless connections. The IMD 16, the programmer 24, the external device 192, and the computing devices 194a-194n may each include, or comprise, one or more processors, such as one or more microprocessors, DSPs, ASICs, FPGAs, programmable logic circuitry, or the like, that may perform various functions and operations, such as those described herein.

The access point 198 may include, or comprise, a device that connects to the network 196 via any of a variety of connections, such as cellular data connection, telephone dial-up, digital subscriber line (DSL), or cable modem connections. In other examples, the access point 198 may be coupled to the network 196 through different forms of connections, including wired or wireless connections. In some examples, the access point 198 may communicate with the programmer 24 and/or the IMD 16. The access point 198 may be co-located with the patient 14 (e.g., within the same room or within the same site as the patient 14) or may be remotely located from the patient 14. For example, the access point 198 may be a home monitor that is located in the patient's home or is portable for carrying with the patient 14.

During operation, the IMD 16 may collect, measure, and store various forms of diagnostic data such as, e.g., RV dysfunction data (including systolic and diastolic dysfunction), LV dysfunction data (including systolic and diastolic dysfunction), QRS duration, and one or diagnostic parameters that may be utilized by the illustrative systems, methods, and processes. In certain cases, the IMD 16 may directly analyze collected diagnostic data and generate any corresponding reports or alerts. In some cases, however, the IMD 16 may send diagnostic data such as the diagnostic parameters, to the programmer 24, the access point 198, and/or the external device 192, either wirelessly or via the access point 198 and the network 196, for remote processing and analysis (e.g., to assess or determine RV or LV dysfunction).

In another example, the IMD 16 may provide the external device 192 with collected diagnostic data or parameters such as, for example, RV dysfunction data, LV dysfunction data, QRS duration, and one or diagnostic parameters, and target CRT settings, transient CRT settings via the access point 198 and the network 196. The external device 192 includes one or more processors 200. In some cases, the external device 192 may request such data, and in some cases, the IMD 16 may automatically or periodically provide such data to the external device 192. Upon receipt of the diagnostic data via the input/output device 202, the external device 192 may be capable of analyzing the data and generating reports, alerts, or other values.

One or more of the computing devices 194a-194n may access the diagnostic data or parameters through the network 196 for use in determining, or assessing, RV and/or LV dysfunction. In some cases, the external device 192 may display the one or more heart metrics indicative of right and/or left ventricular function to a user via the input/output device 202.

In one embodiment, the external device 192 may comprise a secure storage site for diagnostic data or information that has been collected from the IMD 16 and/or the programmer 24. In this embodiment, the network 196 may comprise an Internet network, and trained professionals, such as clinicians, may use the computing devices 194a-194n to securely access stored diagnostic data or parameters such as one or more heart metrics indicative of right ventricular function, one or more heart metrics indicative of left ventricular function, target CRT settings, and transient CRT settings on the external device 192. For example, the trained professionals may utilize secure usernames and passwords to access the stored information on the external device 192. In one embodiment, the external device 192 may be a CareLink server provided by Medtronic, Inc., of Minneapolis, Minnesota.

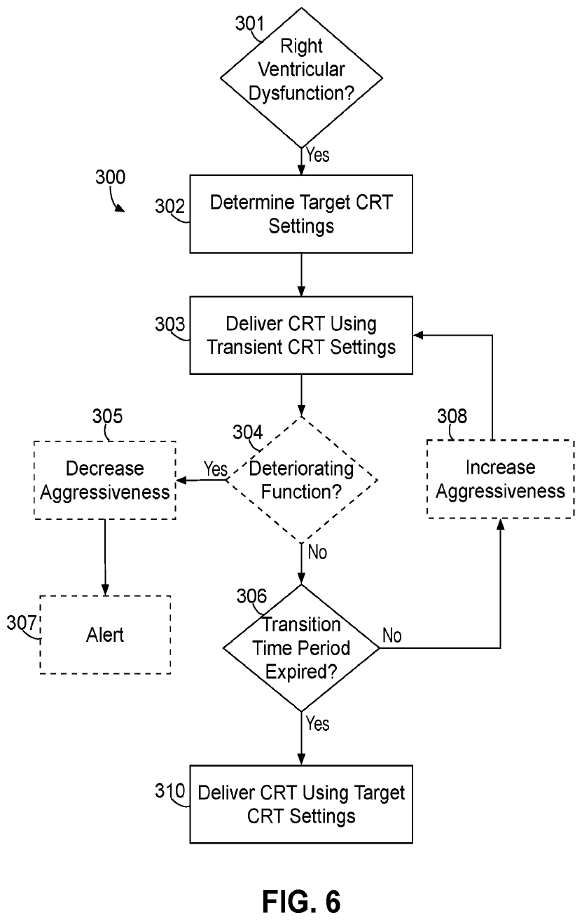

An illustrative method 300 of mitigating right ventricular load during implementation of CRT, for example, using the system and devices of FIGS. 1-5, is depicted in FIG. 6. At the outset, the method 300 may be used, or configured, for patients who have been determined to benefit from CRT. For example, a patient may be afflicted heart failure, impaired left ventricular function, and left bundle branch block, and thus, may be candidate for CRT. It is to be understood that the method 300 may be performed, at least in part, automatically by an implantable medical device and system as described herein with respect to FIGS. 1-5.

The method 300, however, may optionally be directed to only patients who also have right ventricular dysfunction. Thus, the method 300 may optionally include determining whether a patient has right ventricular dysfunction 301 prior to performing the remainder of the method 300. For example, one or more heart metrics may be measured or monitored from the patient, and then used to determine whether a patient has right ventricular dysfunction 301. The one or more heart metrics may be determined using one or more imaging modalities such as magnetic resonance imaging (MRI) or echocardiograms, one or more electrical sensors such as external or internal electrodes measuring cardiac electrical activity (electrocardiograms), and one or more mechanical sensors such as vibration sensors, microphones, and accelerometers.

In one embodiment, a patient's QRS duration (i.e., the duration, or interval, of the QRS complex) or morphology may be determined using cardiac signals measured, or monitored, from the patient using one or more internal or external electrodes, and then used to determine whether a patient has right ventricular dysfunction 301. For example, a change in QRS duration or morphology may be generated by, for example, comparing a previous QRS duration or morphology to a present QRS duration or morphology. The previous QRS duration or morphology may have been measured from a patient prior to having cardiac difficulty. The change in QRS duration or morphology may be represented by a QRS change percentage, which may be compared to a QRS change threshold. If the QRS change percentage is greater than or equal to the QRS change threshold, then it may be determined that the patient has right ventricular dysfunction. The QRS change threshold may, for example, be between about 5% and about 50%. In one embodiment, the QRS change threshold may be 15%. In one embodiment, the QRS change threshold may be greater than or equal to 5%, greater than or equal to 10%, greater than or equal to 12%, greater than or equal to 17%, or greater than or equal to 22%, and/or less than or equal to 50%, less than or equal to 45%, less than or equal to 40%, less than or equal to 35%, less than or equal to 30%, less than or equal to 25%, and less than or equal to 20%. In one embodiment, various imaging technologies such as, e.g., echocardiography or MRI, may be used to determine (e.g., quantify) one or ore both of ejection fraction and stroke volume of the right ventricular, which may be used to determine whether a patient has right ventricular dysfunction 301. In one embodiment, the pulmonary pressure may be measured using by catheterization of the right heart, which may be used to determine whether a patient has right ventricular dysfunction 301.

Additionally, the one or more heart metrics may be measured or monitored from the patient to establish a baseline of the patient's right ventricular functionality, which can then be used during delivery of therapy to determine whether the patient's right ventricle functionality is deteriorating. For example, the baseline of the patient's right ventricular functionality may be compared to the patient's right ventricular functionality during therapy to determine whether the patient's right ventricular functionality is improving or deteriorating.

If the patient is determined to have right ventricular dysfunction 301, then the method 300 may proceed to delivering CRT using transient CRT settings 302. The transient CRT settings may be described as CRT settings that are less aggressive than target CRT settings. The target CRT settings may be configured to optimize left ventricular functionality and restore mechanical synchrony of the patient's heart by electrically activating the heart in a synchronized manner, and the transient CRT settings may be configured to only partially optimize left ventricular functionality and only partially restore mechanical synchrony of the patient's heart. In other words, the target CRT settings may be the ideal CRT settings as determined by a clinician, and the transient settings may be less than the ideal CRT settings such the full functionality restoration of the left ventricle may not be achieved so as to mitigate the burden on the right ventricle.

Each of the target CRT settings and transient CRT settings may include one or more of settings related to the delivery of CRT such as, for example, atrioventricular pacing interval, interventricular pacing interval, left ventricular pacing vector including multisite pacing vectors (in other words, multipoint pacing (MPP)), pacing amplitude, pulses within each pace, and ventricular pacing type such as biventricular pacing, left ventricular-only pacing, traditional myocardial pacing (e.g., pacing pulses delivered directly to muscular tissue of the patient's heart), cardiac conduction system pacing (e.g., pacing pulses delivered directly to one or more portions of the cardiac conduction system such as the left bundle branch, right bundle branch, and/or bundle of HIS), and any combination of thereof (e.g., left bundle branch-optimized CRT and HIS-optimized CRT).

Each of the target CRT settings may be determined prior to determination of the transient CRT settings. For example, during implantation of an implantable medical device, a physician and/or an automated system may configure, or try, each of the CRT settings at different values until determining the target CRT settings that optimize left ventricular functionality and restore mechanical synchrony of the patient's heart by electrically activating the heart in a synchronized manner. In at least one embodiment, an external 12-lead electrocardiogram (ECG) system may be utilized. In at least one embodiment, a system including a plurality of external electrodes (for example, twenty or more electrodes) positioned about the patient's torso may be utilized to monitor a patient's electrical heterogeneity and other metrics during configuration of the CRT settings as described in U.S. Pat. No. 10,064,567 entitled “Systems, Methods, and Interfaces for Identifying Optimal Electrical Vectors” and issued on Sep. 4, 2018, U.S. Pat. No. 9,986,928 entitled “Noninvasive Cardiac Therapy Evaluation” and issued on Jun. 5, 2018, U.S. Pat. No. 9,764,143 entitled “Systems and Methods for Configuration of Interventricular Interval” and issued on Sep. 9, 2017, and U.S. Pat. No. 9,586,050 entitled “Systems and Methods for Configuration of Atrioventricular Interval” and issued on Mar. 7, 2017, each of which are incorporated herein by reference in their entireties.

After the target CRT settings have been determined, the transient CRT settings may be determined based on the target CRT settings. As described herein, the target CRT settings may be configured to optimize left ventricular functionality and restore mechanical synchrony of the patient's heart by electrically activating the heart in a synchronized manner. In contrast, the transient CRT settings may be described as being less aggressive that the target CRT settings thereby mitigating right ventricular load during implementation of CRT, and in particular, during the beginning of the CRT delivery and the transition to the target CRT settings. Because the transient CRT settings are less aggressive that the target CRT settings, the transient CRT settings may be described as being configured to only partially optimize left ventricular functionality and only partially restore mechanical synchrony of the patient's heart. In other words, the transient CRT settings may be described as being CRT settings values lying between intrinsic values and the CRT settings values utilized for target CRT settings. For instance, if a CRT setting may be described in terms of percentage of optimal with the target CRT settings being 100% optimal, and the transient CRT settings being less than 100% optimal such as, for example, 90% optimal, 80% optimal, 70% optimal, 60% optimal, 50% optimal, or 40% optimal. Further, the transient CRT settings may be described as something less than the target CRT settings so as to not completely optimize left ventricular functionality and restore mechanical synchrony of the patient's heart by electrically activating the heart in a synchronized manner. As such, the transient CRT settings may be described as being less than optimal.

The transient CRT settings may utilize, or include, a transient atrioventricular (AV) pacing interval, a transient interventricular (VV) pacing interval, a transient pacing vector, and a transient ventricular pacing type, one or more of which may be adjusted to only partially optimize left ventricular functionality and only partially restore mechanical synchrony of the patient's heart. For example, if the CRT setting is atrioventricular (AV) pacing interval, which is a time period between an atrial intrinsic or paced event and a ventricular paced event, a transient AV pacing interval (i.e., the AV pacing interval of the transient CRT settings) may be greater than, or longer than, a target AV pacing interval (i.e., the AV pacing interval of the target CRT settings) because, for example, a longer AV pacing interval may only partially optimize left ventricular functionality and only partially restore mechanical synchrony of the patient's heart so as to mitigate right ventricular load. In one embodiment, the AV pacing interval of the transient CRT settings may be a selected AV percentage of the atrioventricular pacing interval of the target CRT settings. The selected AV percentage may be between 50% and 150%. In one embodiment, the selected AV percentage is 125%. In one embodiment, the selected AV percentage may be greater than or equal to 50%, greater than or equal to 70%, greater than or equal to 90%, greater than or equal to 100%, greater than or equal to 110%, or greater than or equal to 120%, and/or less than or equal to 150%, less than or equal to 140%, less than or equal to 135%, or less than or equal to 130%.

Further, for example, if the CRT setting is interventricular (VV) pacing interval, which is a time period between a left ventricular paced event and a right ventricular paced event, a transient VV pacing interval (i.e., the VV pacing interval of the transient CRT settings) may be greater than, or longer than, or less than, or shorter than, a target VV pacing interval (i.e., the VV pacing interval of the target CRT settings). The transient VV pacing interval may be greater than or less than the target VV pacing interval depending on the specific patient; in other words, whether the transient VV pacing interval is greater than or less than the target VV pacing interval may vary depending on the patient. Regardless, the transient VV pacing interval will only partially optimize left ventricular functionality and only partially restore mechanical synchrony of the patient's heart so as to mitigate right ventricular load. In one embodiment, the VV pacing interval of the transient CRT settings may be a selected VV percentage of the VV pacing interval of the target CRT settings. The selected VV percentage may be between −100% and 200%.

Further, for example, if the CRT setting is pacing vector, which defines which one or more electrodes are used to deliver paces to the left ventricle, a transient pacing vector (i.e., the pacing vector of the transient CRT settings) may not engage or capture as much of the left ventricle as a target pacing vector (i.e., the pacing vector of the target CRT settings) because, for example, a less engaging pacing vector may only partially optimize left ventricular functionality and only partially restore mechanical synchrony of the patient's heart so as to mitigate right ventricular load. In one embodiment, in a traditional coronary sinus left ventricular lead, the transient left ventricular pacing vector may utilize an apically oriented, or positioned, electrode as the cathode and the target left ventricular pacing vector may utilize a more basal or lateral oriented, or positioned, electrode as the cathode. In one embodiment, in a traditional coronary sinus left ventricular lead, the transient left ventricular pacing vector may utilize a more anterior oriented, or positioned, electrode as the cathode and the target left ventricular pacing vector may utilize a more posterior oriented, or positioned, electrode as the cathode. In other words, the transient left ventricular pacing vector may be configured to deliver pacing to a first location, and the target left ventricular pacing vector may be configured to deliver pacing to a second location different from the first location. The second location may be more posterior and/or basal, lateral, or basolateral than the first location. Conversely, the first location may be more anteroapical than the second location. Additionally, the transient left ventricular pacing vector may utilize a single electrode as the cathode and the target left ventricular pacing vector may utilize multiple electrodes as the cathode (e.g., multipoint pacing). Moreover, in another example, when delivering left bundle branch pacing therapy, the transient left ventricular pacing vector may utilize anodal capture, and the target left ventricular pacing vector may utilize cathodal capture.

Still further, for example, if the CRT setting is ventricular pacing type such as traditional myocardial pacing therapy and cardiac conduction system pacing therapy, a transient ventricular pacing type may be traditional myocardial pacing therapy because, for example, traditional myocardial pacing therapy may provide less synchronized engagement of the left ventricle than the cardiac conduction system pacing therapy so as to only partially optimize left ventricular functionality and only partially restore mechanical synchrony of the patient's heart so as to mitigate right ventricular load.

The transient CRT settings may be delivered 303 to the patient for a transition time period. The transition time period may begin after the pacing device is implanted and CRT therapy is initially delivered to the patient and may end after the expiration of transition time period when the patient is ready to receive the target CRT settings. In particular, for example, the transition time period may be configured to expire after preset time period, or more preferably, configured, or set, to expire when it is determined that the right ventricle of the patient's heart is ready to receive the affects of fully optimized CRT that is provided by the target CRT settings.

As such, the method 300 may check whether the transition time period has expired 306. The transition time period may be between about 2 days and about multiple weeks. In one embodiment, the transition time period is 14 days. In one or more embodiments, the transition time period may be greater than or equal to 2 days, greater than or equal to 4 days, greater than or equal to 7 days, greater than or equal to 10 days, or greater than or equal to 12 days, and/or less than or equal to 90 days, less than or equal to 60 days, less than or equal to 45 days, less than or equal to 30 days, or less than or equal to 14 days. If the transition time period has expired, the method 300 may be configure the pacing device to deliver CRT to use, or according to, the target CRT settings 310. Likewise, If the transition time period has not expired, the method 300 may continue delivering CRT using, or according to, the transient CRT settings 302.

Optionally, as indicated by the dashed line boxes in FIG. 6, the method 300 may increase or decrease the aggressiveness of the transient CRT settings during the transition time period. For instance, the method 300 may include determining whether the patient's ventricular function such as, for example, right ventricular function, is deteriorating 304 based on one or more heart metrics, and decreasing the transient CRT settings to be less aggressive 305 in response to, or if, it is determined that the patient's ventricular function is deteriorating based on the one or more heart metrics. The one or more heart metrics may be similar to or the same those one or more metrics utilized to determine right ventricular dysfunction 301. For example, QRS duration may be evaluated, or assessed, to determine that the patient's right ventricular function is deteriorating 304. In particular, for example, a patient's present QRS duration may be compared to the patient's baseline QRS duration that was measured prior to receiving CRT, and if the patient's present QRS duration is worse than the patient's baseline QRS duration, then it may be determined that the patient's right ventricular function is deteriorating 304. Additionally, for example, a patient's present QRS duration may be compared to a threshold QRS duration indicative of right ventricular dysfunction, and if the patient's present QRS duration is greater than or equal to the threshold QRS duration, then it may be determined that the patient's right ventricular function is deteriorating 304. Also, for example, patient reported symptoms such as shortness of breath may be reported by a patient (e.g., through a user interface device like a smartphone, directly to a physician, etc.), and if the patient reported symptoms are indicative of right ventricular dysfunction such as shortness of breath, then it may be determined that the patient's right ventricular function is deteriorating 304.

As described herein, if it is determined that the patient's right ventricular function is deteriorating 304, then the aggressiveness of the transient CRT settings may be decreased 305. More specifically, for instance, one or more of the transient CRT settings may be adjusted to be less aggressive in applying the CRT. For example, the transient AV pacing delay may be increased by an increment such as 25 milliseconds (ms) to adjust the transient settings to be less aggressive. Further, for example, the transient VV pacing delay may be increased or decreased by a value such as 10 ms to adjust the transient settings to be less aggressive. Still further, for example, in situations where more than two pacing vectors exist, a pacing vector that is less aggressive (e.g., less effective in engaging left ventricular tissue) than the present pacing vector may be selected when adjusting the transient settings to be less aggressive.

Additionally, if the aggressiveness of the transient CRT settings is decreased, the method 300 may issue an alert 307, for example, to one or more of the patient, a clinician, and an external server. For example, an alert may be delivered wirelessly to a patient's smartphone. Further, for example, an alert may be transmitted to an external server storing medical records using wireless and/or wireless networks and presented to the patient's clinician.

In one embodiment, once it is determined that the patient's right ventricular function is deteriorating 304, the aggressiveness of the transient CRT settings is decreased 305, and the alert is issued 307, CRT therapy may continue to be delivered according to the transient CRT settings with decreased aggressiveness until the patient is evaluated by a clinician. In another embodiment, once it is determined that the patient's right ventricular function is deteriorating 304, the aggressiveness of the transient CRT settings is decreased 305, and the alert is issued 307, the method 300 may return to process 303 and continue to wait until the expiration of transition time period 306 until adjusting the CRT to the target CRT settings.

As noted above, the method 300 may also increase the aggressiveness of the transient CRT settings during the transition time period. For instance, the method 300 may periodically determine whether the transition time period has expired 306 and, if the transition time period has not expired 306 (and no deteriorating ventricular function as been determined), then the method 300 may adjust the transient CRT settings to be more aggressive 308. More specifically, for instance, one or more of the transient CRT settings may be adjusted to be more aggressive in applying the CRT. For example, the transient AV pacing delay may be decreased by a decrement such as 25 ms to adjust the transient settings to be more aggressive. Further, for example, the transient VV pacing delay may be increased or decreased by a value such as 10 ms to adjust the transient settings to be more aggressive. Still further, for example, in situations where more than 2 pacing vectors exist, a pacing vector that is more aggressive (e.g., more effective in engaging left ventricular tissue) than the present pacing vector may be selected when adjusting the transient CRT settings to be more aggressive.

By increasing the aggressiveness of the transient CRT settings 308 over the transition time period, the transient CRT settings may gradually approach the target CRT settings thereby easing the transition from the transient CRT settings to the target CRT settings. It is to be understood that the adjustments to one or more of the transient CRT settings may be adjusted according to one or more gradual functions. Further, when using fixed values to increase the aggressiveness of the transient CRT settings as previously described herein, the adjustments to one or more of the transient CRT settings may be adjusted according to one or more step-wise, or stepped, gradual functions. Thus, the transient CRT settings may be adjusted over time according to a series of incremental steps or smooth transitions, which may be according to a gradual function.

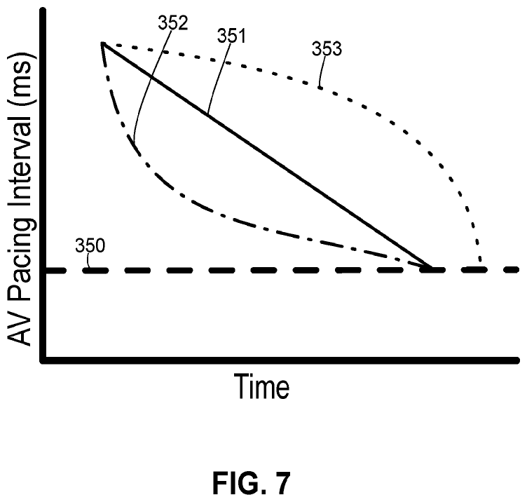

For instance, a graph of a transient CRT setting over time is depicted in FIG. 7.

Although the transient CRT setting depicted along the y-axis is AV pacing interval, it is to be understood that any transient CRT setting may be utilized in a similar fashion. As shown, the target AV pacing interval is represented by dashed lined 350 and three different gradual AV pacing interval functions 351, 352, 353 are depicted as decreasing over a transition time period until reaching the target AV pacing interval 350. Each of the three gradual AV pacing interval functions 351, 352, 353 decreases AV pacing interval, thereby increasing aggressiveness thereof, utilizing different functions. In particular, the gradual AV pacing interval function 351 is a linear function (e.g., having a constant negative slope), the gradual AV pacing interval function 352 is a power or quadratic function where the negative slope decreases over time, and the gradual AV pacing interval function 353 is a power or quadratic function where the negative slope increases over time.

Additionally, it is to be understood that the aggressiveness of more than a single transient CRT setting may be increased during the transition time period. For example, the aggressiveness of both of AV pacing interval and VV pacing interval may be increased during the transition time period, which may be represented in a three-dimensional graph where time is along the x-axis, AV pacing interval is along the y-axis, and VV pacing interval is along the z-axis.

Furthermore, when a CRT setting is effectively a binary decision, or choice, such as between traditional myocardial pacing therapy and cardiac conduction system pacing therapy, changes between such therapies may occur once during the transition time period or only at the expiration of the transition time period, which, for example, may depend on the magnitude of right ventricular load such change would incur.

### EXAMPLES

Example Ex1: An implantable medical device comprising:
an electrode apparatus comprising one or more electrodes configured to deliver cardiac resynchronization therapy (CRT) to a patient's heart using CRT settings and to sense electrical activity of the patient's heart; and
a computing apparatus operably coupled to the electrode apparatus and comprising processing circuitry, the computing apparatus configured to:
   provide target CRT settings configured to optimize left ventricular functionality and restore mechanical synchrony of the patient's heart by electrically activating the heart in a synchronized manner; and
   mitigate right ventricular load during implementation of CRT by delivering CRT to the patient's heart using the one or more electrodes according to transient CRT settings that are less aggressive than the target CRT settings to partially optimize left ventricular functionality.

Example Ex2: An implantable medical device comprising:
an electrode apparatus comprising one or more electrodes configured to deliver cardiac resynchronization therapy (CRT) to a patient's heart using CRT settings and to sense electrical activity of the patient's heart; and
a computing apparatus operably coupled to the electrode apparatus and comprising processing circuitry, the computing apparatus configured to:
   deliver CRT according to transient CRT settings to the patient's heart using the one or more electrodes, wherein the transient CRT settings are less aggressive than target CRT settings to partially optimize left ventricular functionality to mitigate right ventricular load; and
   transition from the transient CRT settings to the target CRT settings over a transition time period.

Example Ex3: A method comprising:
delivering cardiac resynchronization therapy (CRT) to a patient's heart using one or more electrodes of an implantable medical device;
providing target CRT settings configured to optimize left ventricular functionality and restore mechanical synchrony of the patient's heart by electrically activating the heart in a synchronized manner; and
mitigating right ventricular load during implementation of CRT by delivering CRT to the patient's heart using the one or more electrodes according to transient CRT settings that are less aggressive than the target CRT settings to partially optimize left ventricular functionality.

Example Ex4: A method comprising:
delivering cardiac resynchronization therapy (CRT) to a patient's heart using one or more electrodes of an implantable medical device according to transient CRT settings to the patient's heart using the one or more electrodes, wherein the transient CRT settings are less aggressive than target CRT settings to partially optimize left ventricular functionality to mitigate right ventricular load; and
transitioning from the transient CRT settings to the target CRT settings over a transition time period.

Example Ex5: The device as in Examples Ex1-Ex2 or the method as in Examples Ex3-Ex4, wherein each of the target and transient CRT settings comprises one or more of an atrioventricular pacing interval and an interventricular pacing interval, wherein one or more of the atrioventricular pacing interval and interventricular pacing interval of the transient CRT settings are a selected percentage of one or more of the atrioventricular pacing interval and interventricular pacing interval, respectively, of the target CRT settings.

Example Ex6: The device or method as in Example Ex5, wherein the selected percentage is greater than or equal to 125%.

Example Ex7: The device or method as in any one of Examples Ex1-Ex6, wherein each of the target and transient CRT settings comprises a left ventricular pacing vector, wherein the left ventricular pacing vector of the transient CRT settings is different and less aggressive than the left ventricular pacing vector of the target CRT settings.

Example Ex8: The device or method as in Example Ex7, wherein the left ventricular pacing vector of the transient CRT settings is configured to deliver pacing to a first location and the left ventricular pacing vector of the target CRT settings is configured to deliver pacing to a second location. In one example, the second location is closer to the basal region of the left ventricle than the first location. In one example, the first location is closer to the apical region of the left ventricle than the second location. In one example, the first location is closer to the anterior region of the left ventricle than the second location. In one example, the second location is closer to the posterior region of the left ventricle than the first location. In one example, the first location is closer to the anteroapical region of the left ventricle than the second location. In one example, the second location is closer to the posterolateral or basolateral region of the left ventricle than the first location.

Example Ex9: The device or method as in any one of Examples Ex1-Ex8, wherein each of the target and transient CRT settings comprises a ventricular pacing type, wherein the ventricular type of the transient CRT settings is left-ventricular only pacing and the ventricular pacing type of the target CRT settings is biventricular pacing.

Example Ex10: The device or method as in any one of Examples Ex1-Ex9, wherein each of the target and transient CRT settings comprises a ventricular pacing type, wherein the ventricular type of the transient CRT settings comprises at least cardiac conduction system pacing therapy and the ventricular pacing type of the target CRT settings comprises myocardial pacing therapy.

Example Ex11: The device or method as in any one of Examples Ex1, Ex3, and Ex5-Ex10, wherein mitigating right ventricular load during implementation of CRT by delivering CRT to the patient's heart using the one or more electrodes according to transient CRT settings that are less aggressive than the target CRT settings to partially optimize left ventricular functionality comprises delivering CRT to the patient's heart using the electrode one or more electrodes according to transient CRT settings for a transition time period prior to delivering CRT to the patient's heart using the one or more electrodes according to target CRT settings,
wherein the computing apparatus is further configured to execute or the method further comprises delivering CRT to the patient's heart using the one or more electrodes according to target CRT settings following expiration of the transition time period.

Example Ex12: The device or method as in Examples Ex2, Ex 4, and Ex11, wherein the transition time period is greater than or equal to one week.

Example Ex13: The device or method as in Example Ex11, wherein delivering CRT to the patient's heart using the one or more electrodes according to transient CRT settings for a transition time period prior to delivering CRT to the patient's heart using the one or more electrodes according to target CRT settings comprises gradually increasing aggressiveness of the transient CRT settings over the transition time period.

Example Ex14: The device as in Example Ex2 or the method as in Example Ex4, wherein transitioning from the transient CRT settings to the target CRT settings over a transition time period comprises gradually increasing aggressiveness of the transient CRT settings over the transition time period until reaching the target CRT settings.

Example Ex15: The device or method as in any one of Examples Ex1-Ex14, wherein the computing apparatus is further configured to execute or the method further comprises:
monitoring one or more heart metrics indicative of right ventricular function using the one or more electrodes;
determining that the patient's right ventricular function is deteriorating based on the one or more heart metrics; and
adjusting the transient CRT settings to be less aggressive.

Example Ex16: The device or method as in Example Ex15, wherein the one or more heart metrics comprises QRS duration.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described methods, processes, and techniques, including those attributed to the IMD 16, the programmer 24, the external device 192, and computing devices 194n, may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. The terms “computing apparatus,” “controller” “module,” “processor,” or “processing circuitry” may generally refer to any of the foregoing logic circuitry, alone or in combination with other logic circuitry, or any other equivalent circuitry. Also, the techniques could be fully implemented in one or more circuits or logic elements. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

All references and publications cited herein are expressly incorporated herein by reference in their entirety for all purposes, except to the extent any aspect directly contradicts this disclosure.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

As used herein, the term “configured to” may be used interchangeably with the terms “adapted to” or “structured to” unless the content of this disclosure clearly dictates otherwise.

The singular forms “a,” “an,” and “the” encompass embodiments having plural referents unless its context clearly dictates otherwise.

As used herein, “have,” “having,” “include,” “including,” “comprise,” “comprising” or the like are used in their open-ended sense, and generally mean “including, but not limited to.” It will be understood that “consisting essentially of,” “consisting of,” and the like are subsumed in “comprising,” and the like.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

1. An implantable medical device comprising:
an electrode apparatus comprising one or more electrodes configured to deliver cardiac resynchronization therapy (CRT) to a patient's heart using CRT settings and to sense electrical activity of the patient's heart; and
a computing apparatus operably coupled to the electrode apparatus and comprising processing circuitry, the computing apparatus configured to:
deliver CRT according to transient CRT settings to the patient's heart using the one or more electrodes, wherein the transient CRT settings are less aggressive than target CRT settings to partially optimize left ventricular functionality to mitigate right ventricular load; and
transition from the transient CRT settings to the target CRT settings over a transition time period.

2-4. (canceled)

5. The device of claim 1, wherein each of the target and transient CRT settings comprises one or more of an atrioventricular pacing interval and an interventricular pacing interval, wherein one or more of the atrioventricular pacing interval and interventricular pacing interval of the transient CRT settings are a selected percentage of one or more of the atrioventricular pacing interval and interventricular pacing interval, respectively, of the target CRT settings.

6. The device of claim 5, wherein the selected percentage is greater than or equal to 125%.

7. The device of claim 1, wherein each of the target and transient CRT settings comprises a left ventricular pacing vector, wherein the left ventricular pacing vector of the transient CRT settings is different and less aggressive than the left ventricular pacing vector of the target CRT settings.

8. The device of claim 7, wherein the left ventricular pacing vector of the transient CRT settings is configured to deliver pacing to a first location and the left ventricular pacing vector of the target CRT settings is configured to deliver pacing to a second location, wherein the first location is closer to the anteroapical region of the left ventricle than the second location.

9. The device of claim 1, wherein each of the target and transient CRT settings comprises a ventricular pacing type, wherein the ventricular type of the transient CRT settings is left-ventricular only pacing and the ventricular pacing type of the target CRT settings is biventricular pacing.

10. The device of claim 1, wherein each of the target and transient CRT settings comprises a ventricular pacing type, wherein the ventricular type of the transient CRT settings comprises at least cardiac conduction system pacing therapy and the ventricular pacing type of the target CRT settings comprises myocardial pacing therapy.

11. (canceled)

12. The device of claim 1, wherein the transition time period is greater than or equal to one week.

13. The device of claim 1, wherein delivering CRT to the patient's heart according to transient CRT settings for a transition time period prior to delivering CRT to the patient's heart according to target CRT settings comprises gradually increasing aggressiveness of the transient CRT settings over the transition time period.

14. The device of claim 1, wherein transitioning from the transient CRT settings to the target CRT settings over a transition time period comprises gradually increasing aggressiveness of the transient CRT settings over the transition time period until reaching the target CRT settings.

15. The device of claim 1, wherein the computing apparatus is further configured to:
monitor one or more heart metrics indicative of right ventricular function;
determine that the patient's right ventricular function is deteriorating based on the one or more heart metrics; and
adjust the transient CRT settings to be less aggressive.

16. The device of claim 15, wherein the one or more heart metrics comprises QRS duration.

17. A method comprising:
delivering cardiac resynchronization therapy (CRT) according to transient CRT settings to a patient's heart using one or more electrodes of an implantable medical device, wherein the transient CRT settings are less aggressive than target CRT settings to partially optimize left ventricular functionality to mitigate right ventricular load; and
transitioning from the transient CRT settings to the target CRT settings over a transition time period.

18. The method of claim 17, wherein each of the target and transient CRT settings comprises one or more of an atrioventricular pacing interval and an interventricular pacing interval, wherein one or more of the atrioventricular pacing interval and interventricular pacing interval of the transient CRT settings are a selected percentage of one or more of the atrioventricular pacing interval and interventricular pacing interval, respectively, of the target CRT settings.

19. The method of claim 18, wherein the selected percentage is greater than or equal to 125%.

20. The method of claim 17, wherein each of the target and transient CRT settings comprises a left ventricular pacing vector, wherein the left ventricular pacing vector of the transient CRT settings is different and less aggressive than the left ventricular pacing vector of the target CRT settings.

21. The method of claim 20, wherein the left ventricular pacing vector of the transient CRT settings is configured to deliver pacing to a first location and the left ventricular pacing vector of the target CRT settings is configured to deliver pacing to a second location, wherein the first location is closer to the anteroapical region of the left ventricle than the second location.

22. The method of claim 17, wherein each of the target and transient CRT settings comprises a ventricular pacing type, wherein the ventricular type of the transient CRT settings is left-ventricular only pacing and the ventricular pacing type of the target CRT settings is biventricular pacing.

23. The method of claim 17, wherein each of the target and transient CRT settings comprises a ventricular pacing type, wherein the ventricular type of the transient CRT settings comprises at least cardiac conduction system pacing therapy and the ventricular pacing type of the target CRT settings comprises myocardial pacing therapy.

24. The method of claim 17, wherein the transition time period is greater than or equal to one week.

25. The method of claim 17, wherein delivering CRT to the patient's heart according to transient CRT settings for a transition time period prior to delivering CRT to the patient's heart according to target CRT settings comprises gradually increasing aggressiveness of the transient CRT settings over the transition time period.

26. The method of claim 17, wherein transitioning from the transient CRT settings to the target CRT settings over a transition time period comprises gradually increasing aggressiveness of the transient CRT settings over the transition time period until reaching the target CRT settings.

27. The method of claim 17, wherein the method further comprises:
monitoring one or more heart metrics indicative of right ventricular function;
determining that the patient's right ventricular function is deteriorating based on the one or more heart metrics; and
adjusting the transient CRT settings to be less aggressive.

28. The method of claim 27, wherein the one or more heart metrics comprises QRS duration.

29. An implantable medical device comprising:
an electrode apparatus comprising one or more electrodes configured to deliver cardiac resynchronization therapy (CRT) to a patient's heart using CRT settings and to sense electrical activity of the patient's heart; and
a computing apparatus operably coupled to the electrode apparatus and comprising processing circuitry, the computing apparatus configured to:
provide target CRT settings configured to optimize left ventricular functionality and restore mechanical synchrony of the patient's heart by electrically activating the heart in a synchronized manner; and
mitigate right ventricular load during implementation of CRT by delivering CRT to the patient's heart using the one or more electrodes according to transient CRT settings that are less aggressive than the target CRT settings to partially optimize left ventricular functionality.

30. The device of claim 29, wherein mitigating right ventricular load during implementation of CRT by delivering CRT to the patient's heart using the one or more electrodes according to transient CRT settings that are less aggressive than the target CRT settings to partially optimize left ventricular functionality comprises delivering CRT to the patient's heart using the one or more electrodes according to transient CRT settings for a transition time period prior to delivering CRT to the patient's heart using the one or more electrodes according to target CRT settings,
wherein the computing apparatus is further configured to deliver CRT to the patient's heart using the one or more electrodes according to target CRT settings following expiration of the transition time period.
